(19) United States
(12) Patent Application Publication
(10) Pub. No.: US 2015/0202399 A1
(43) Pub. Date: Jul. 23, 2015

(54) PLATFORM FOR OXYGEN GENERATION AND DELIVERY

(71) Applicant: PURDUE RESEARCH FOUNDATION, West Lafayette, IN (US)

(72) Inventors: Babak Ziaie, West Lafayette, IN (US); Manuel Ochoa, West Lafayette, IN (US); Charilaos Mousoulis, Lafayette, IN (US)

(73) Assignee: PURDUE RESEARCH FOUNDATION, West Lafayette, IN (US)

(21) Appl. No.: 14/602,203

(22) Filed: Jan. 21, 2015

Related U.S. Application Data
(60) Provisional application No. 61/929,931, filed on Jan. 21, 2014.

(51) Int. Cl. A61M 16/10; A61M 16/06; A61M 16/08; A61M 35/00

(52) U.S. Cl. CPC A61M 16/101; A61M 35/00; A61M 16/0672; A61M 16/0875; A61M 2202/0208; A61M 2207/00; A61M 2209/088

(57) ABSTRACT

A platform for oxygen generation and delivery is disclosed. The platform includes a hydrophobic substrate with at least one hydrophilic region permeable to gas flow formed thereon having an oxygen generating compound embedded in the at least one hydrophilic region. The platform further includes a microfluidic network with an inlet and an outlet bonded to the hydrophobic substrate with at least one fluid exchange region fluidly coupled to the inlet and the outlet and substantially matching the least one hydrophilic region. The microfluidic network is configured to receive an oxygen rich fluid at the inlet, communicate the oxygen rich fluid to the at least one fluid exchange region to mix with the oxygen generating compound, causing a chemical reaction resulting in formation of oxygen and a chemical byproduct, and communicate the chemical byproduct to the outlet, where the oxygen is permeated out of the at least one hydrophilic region.

### CROSS-REFERENCE TO RELATED APPLICATIONS

The present U.S. patent application is related to and claims the priority benefit of U.S. Provisional Patent Application Ser. No. 61/929,931, filed Jan. 21, 2014, the contents of which is hereby incorporated by reference in its entirety into the present disclosure.

### STATEMENT REGARDING GOVERNMENT FUNDING

This invention was made with government support under EFRI1240443 awarded by the National Science Foundation. The government has certain rights in the invention.

### TECHNICAL FIELD

The present disclosure generally relates to oxygen generation and delivery, and in particular to oxygen generation and delivery in wound treatment and life support.

### BACKGROUND

On demand oxygen generation and delivery is critical in medicine, aviation, and industrial applications. Most methods currently employ oxygen tanks (liquid or gas phase) that are bulky, hazardous, and expensive. A low cost, on-demand oxygen generating platform would therefore be of immense value. In medicine for example, suboptimal oxygenation of the wound bed is a major healing inhibitor in chronic wounds. Unlike acute injuries that receive sufficient oxygen via a working blood vessel network, chronic wounds often suffer from an irregular vasculature structure incapable of providing sufficient oxygen for tissue growth. While the lack of oxygen may trigger vascular regeneration, the severity and depth of wounds can prevent adequate regeneration, causing wound ischemia.

Modern medical treatment of hypoxic chronic wounds typically employs hyperbaric oxygen therapy, which requires bulky equipment and often exposes large areas of the body to unnecessarily elevated oxygen concentrations that can damage healthy tissue. Hence, such methods require very careful and periodic oxygen administration to avoid hyper-oxygenation of tissue surrounding the wound.

Therefore, there is an unmet need for a device for treatment of such wounds from the use of a localized method for oxygen delivery with improved precision.

### SUMMARY

A platform for oxygen generation and delivery is disclosed. The platform includes a hydrophobic substrate with at least one hydrophilic region permeable to gas flow formed thereon having an oxygen generating compound embedded in the at least one hydrophilic region. The platform further includes a microfluidic network with an inlet and an outlet bonded to the hydrophobic substrate with at least one fluid exchange region fluidly coupled to the inlet and the outlet and substantially matching the least one hydrophilic region. The microfluidic network is configured to receive an oxygen rich fluid at the inlet, communicate the oxygen rich fluid to the at least one fluid exchange region to mix with the oxygen generating compound, causing a chemical reaction resulting in formation of oxygen and a chemical byproduct, and communicate the chemical byproduct to the outlet, where the oxygen is permeated out of the at least one hydrophilic region.

A method of healing wounds is also disclosed. The method includes placing a flexible wound healing device on a wounded tissue. The flexible wound healing device is configured to generate oxygen at higher concentrations than present in air. The method also includes injecting an oxygen-rich fluid into the flexible wound healing device. The flexible wound healing device includes a hydrophobic substrate with at least one hydrophilic region formed thereon and positioned over the wounded tissue and which is permeable to gas flow and having an oxygen generating compound embedded in the at least one hydrophilic region. The flexible wound healing device further includes a microfluidic network with an inlet and an outlet bonded to the hydrophobic substrate with at least one fluid exchange region fluidly coupled to the inlet and the outlet and which is substantially matching the least one hydrophilic region. The microfluidic network is configured to receive the oxygen rich fluid at the inlet, communicate the oxygen rich fluid to the at least one fluid exchange region to mix with the oxygen generating compound, causing a chemical reaction resulting in formation of oxygen and a chemical byproduct, and communicate the chemical byproduct to the outlet, where the oxygen is permeated out of the at least one hydrophilic region.

A method of fabricating a platform for oxygen generation and delivery is also disclosed. The method includes laser-patterning a hydrophobic substrate to produce at least one hydrophilic region. The method further includes embedding an oxygen generating compound in the at least one hydrophilic region. The method also includes forming a microfluidic network having an inlet, an outlet and at least one fluid exchange region in fluid communication with the inlet and the outlet. The method further includes bonding the microfluidic network to the hydrophobic substrate such that the at least one fluid exchange region is positioned over the hydrophilic region. The microfluidic network is configured to receive an oxygen rich fluid at the inlet, communicate the oxygen rich fluid to the at least one fluid exchange region to mix with the oxygen generating compound, causing a chemical reaction resulting in formation of oxygen and a chemical byproduct, and communicate the chemical byproduct to the outlet, where the oxygen is permeated out of the at least one hydrophilic region.

### BRIEF DESCRIPTION OF THE FIGURES

FIG. 1a is a cross-sectional view of a wound healing platform, including a pumping device, and a flexible wound healing device placed over a wounded tissue.

FIG. 1b is a cross-sectional view of the flexible wound healing device of FIG. 1a, including a microfluidic network and a substrate system.

FIG. 1c is a cross-sectional view of the microfluidic network of FIG. 1b.

FIG. 1d is a cross-sectional view of the substrate system of FIG. 1b.

FIG. 1e is an alternative embodiment of a breathing apparatus that can be used in acute or chronic oxygen generation systems.

FIGS. 2a-2g are cross-sectional views of a process for fabricating the flexible wound healing device of FIG. 1a.

FIG. 3a is a test polydimethylsiloxane (PDMS)-parchment paper platform for characterizing various aspects of the flexible wound healing device.

FIGS. 3b and 3c are photographs of the fabricated test platform of FIG. 3a.

FIGS. 4a and 4b are scanning electron microscope images of a catalyst in an aqueous solution and a catalyst that formed based on a chemical reaction.

FIG. 5 is a graph of pressure measured in torr for a bond between the microfluidic network and substrate system of FIG. 1b to fail for different configurations of the microfluidic network.

FIG. 6 is a graph of pressure measured in torr vs. flowrate in μL/min as a measure of permeability for the substrate system of FIG. 1b.

FIG. 7 is a graph of oxygen percentage next to various areas of the substrate system of FIG. 1b.

FIG. 8 is a graph of oxygen generation measured in μL/mm² vs. time measured in minutes for long term oxygen generation (30 hours).

FIG. 9 is a graph of oxygen levels in air measured in percentage vs. distance from the substrate system of FIG. 1b for different flowrates of an oxygen generating fluid.

FIG. 10a is a graph of fluorescence intensity for metabolic rates of 3T3 cells that are seeded on the culture dish as a control and that of the cells seeded on the substrate system of FIG. 1b where in one case there is a catalyst and in one case there is no catalyst.

FIG. 10b is a graph of fluorescence intensity for metabolic rates of 3T3 cells of the cells seeded on the substrate system of FIG. 1b where in one case there is an oxygen generating fluid and in another cases there is no oxygen generating fluid.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

As a low-cost alternative for continuous oxygen delivery, presented herein is a novel, inexpensive, paper-based, biocompatible, and flexible oxygen generating platform for locally generating and delivering oxygen to selected hypoxic regions. The platform takes advantage of recent developments in the fabrication of flexible microsystems including the incorporation of paper as a substrate and the use of inexpensive laser machining. Together they enable the development of low-cost patches with customized, wound-specific oxygen generation regions for use and benefits that include the following fields: treatment of hypoxic tissues such as cardiac, skin (wound), and tumors, life support applications in respiratory failure such as emergency and intensive care, and life support for aviation and other industries that rely on carrying bulky oxygen bottles.

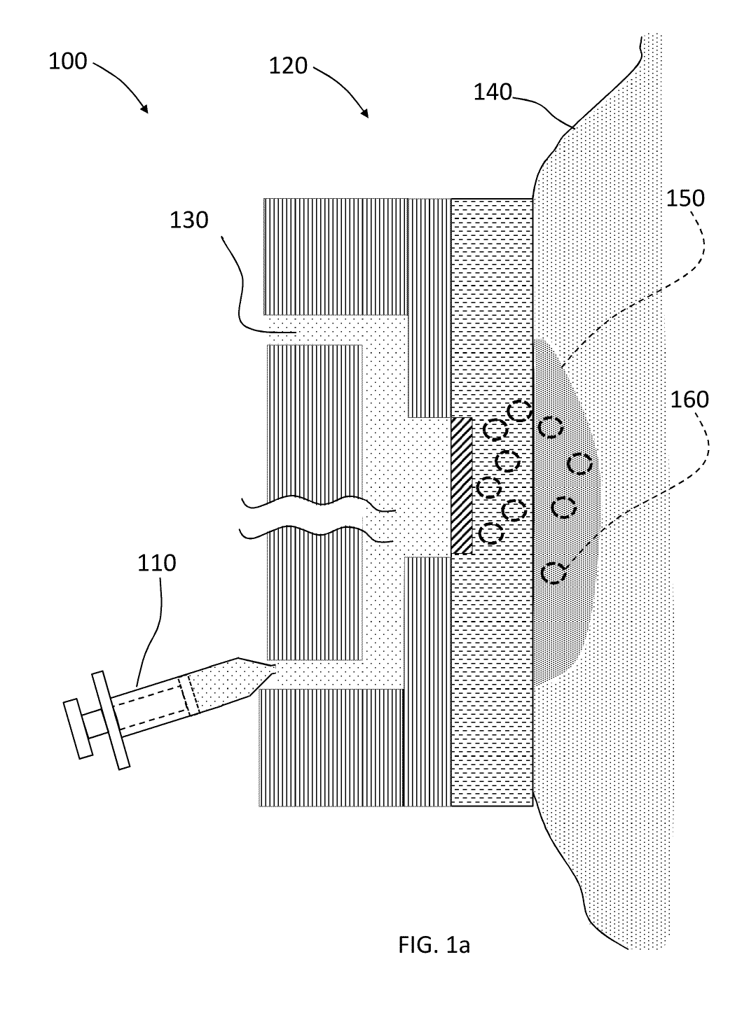

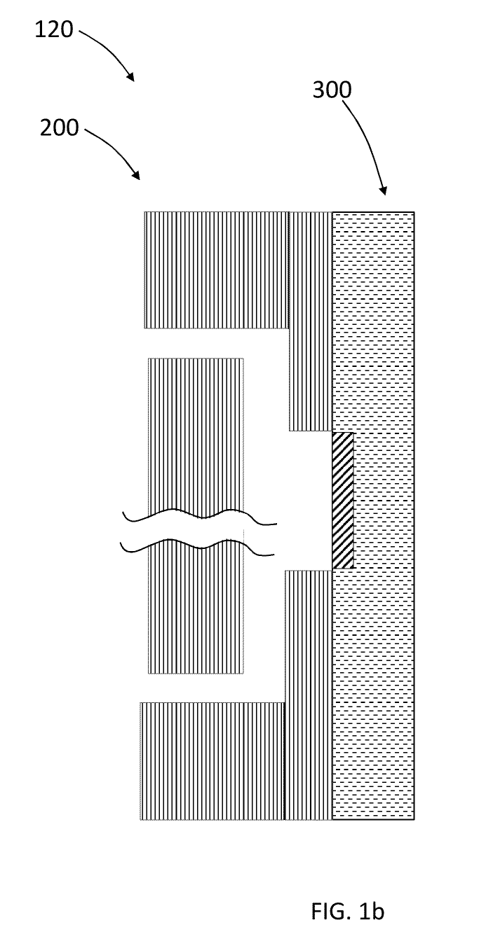

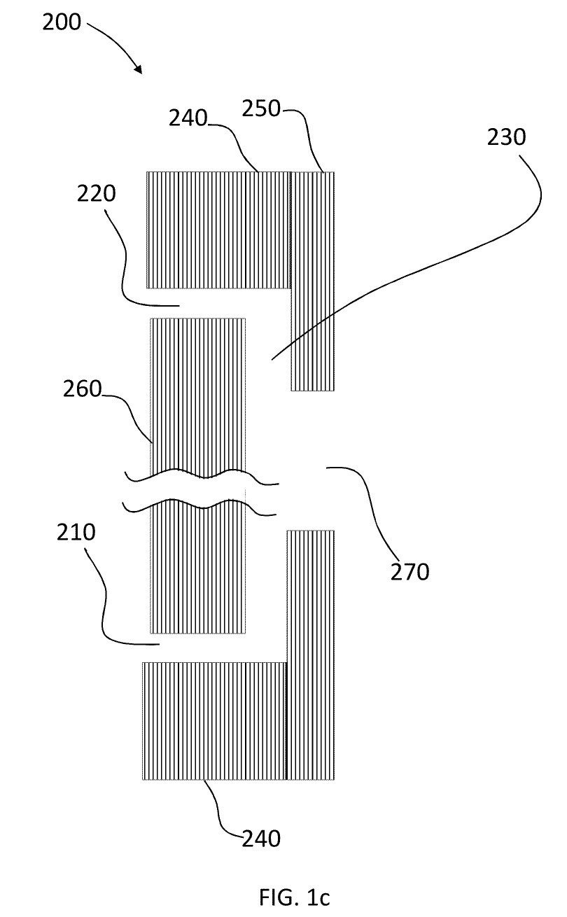

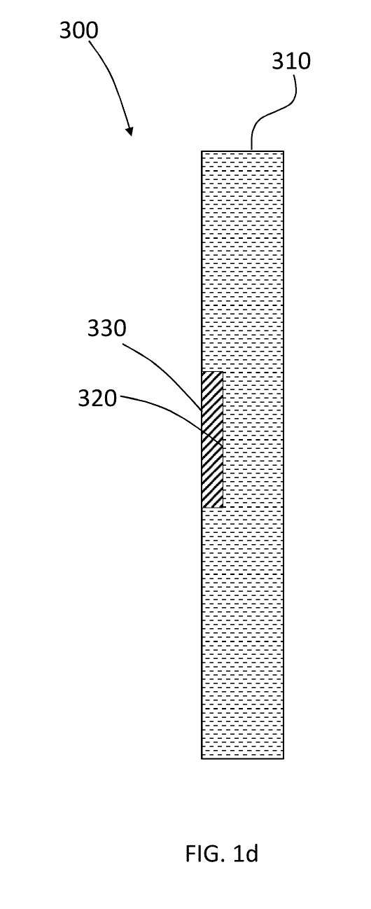

As illustrated in FIG. 1a, a cross sectional view of a platform 100 for wound healing is depicted. The platform includes a pumping device 110, e.g., a syringe, and a flexible wound healing device 120 disposed on a wounded tissue 150 (to be distinguished from healthy tissue 140). An oxygen-rich fluid 130 is shown to be pumped from the pumping device 110 into the wound healing device 120 generating oxygen (O₂) 160 near and into the wounded tissue 150. The oxygen-rich fluid 130 enters the wound healing device 120 at an inlet 210 (as described later with respect to FIG. 1c) and exists at an outlet 220 (as described later with respect to FIG. 1c).

As described above, wounded tissue 150 is hypoxic (i.e., lacks sufficient oxygen), thereby causing a prolonged healing. By generating oxygen 160, wound healing can be accelerated, while controlling the rate at which oxygen is generated, thereby preventing damage to the wounded tissue 150 or the healthy tissue 140.

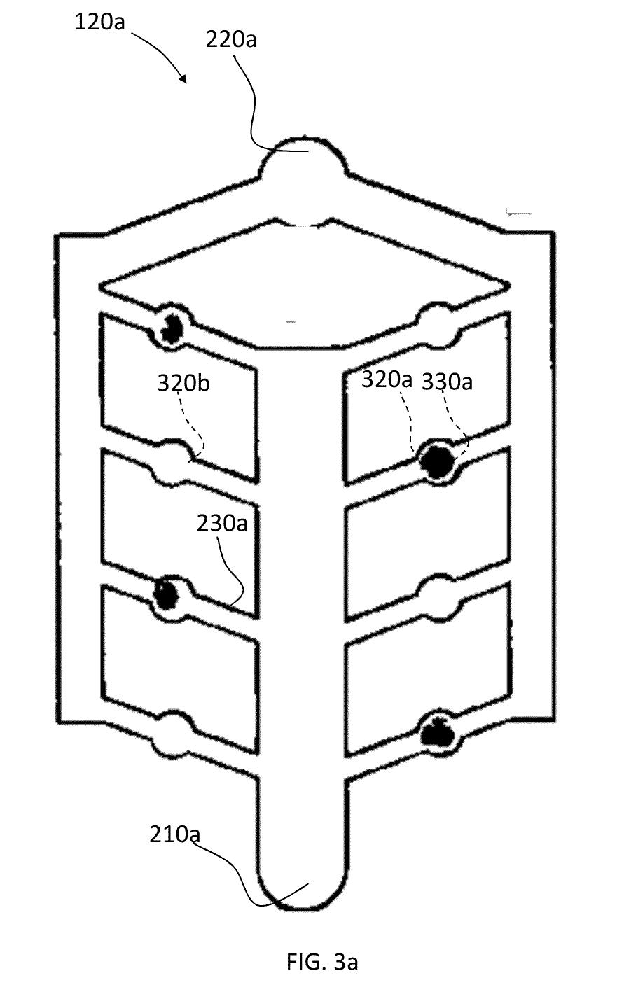

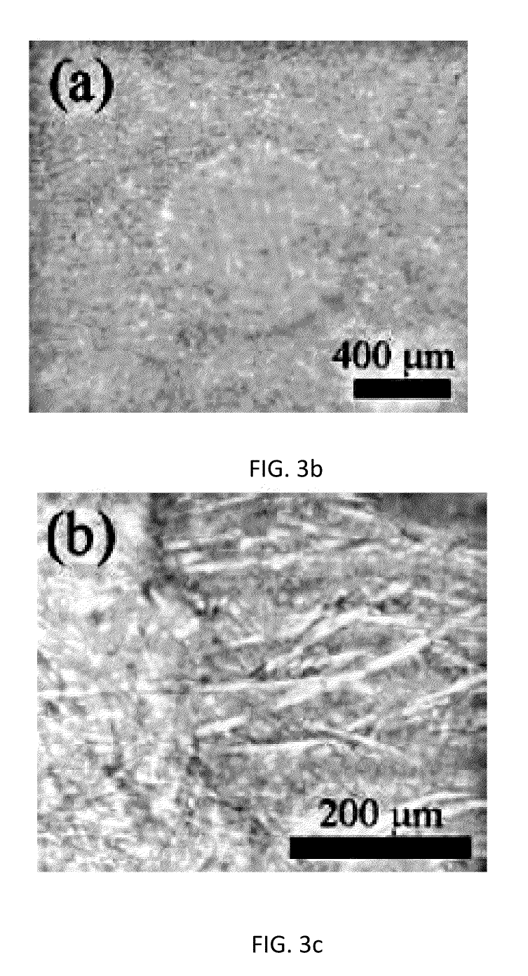

Referring to FIG. 1b, the flexible wound healing device 120 is depicted. The wound healing device 120 includes a flexible microfluidic network 200 and a flexible substrate system 300. The microfluidic network 200 is disposed on the substrate system 300 in a manner as described further below. The microfluidic network 200 can be made from an inert, non-toxic, non-flammable, and preferably clear polymeric compound, e.g., polydimethylsiloxane (or PDMS), or other varieties of silicone. Referring to FIG. 1c, a cross sectional view of the microfluidic network 200 is provided. The system 200 includes sidewalls 240, a base portion 250, and a top portion 260. The system 200 is shown with broken lines to indicate the length of the system 200 may be longer than shown (reference is made to FIG. 3a). These components provide the inlet 210, the outlet 220, and a microchannel 230. The base portion 250 includes an opening 270 (also referred herein as a fluid exchange region) in fluid communication with the inlet 210 and the outlet 220 through the fluid microchannels 230 to allow fluid communication between the microfluidic network 200 and the substrate system 300, where the fluid exchange region 270 is substantially matching, i.e., within about 10% of the area covered by the opening 320 of the substrate system 300 (see FIG. 1d). The components of the system 200 can be made separately and welded together or made in a mold one or more pieces.

Referring to FIG. 1d, a cross sectional view of the substrate system 300 is depicted. The substrate system 300 includes a substrate and an opening 320 (also referred herein as catalyst spots or just simply spots) disposed on the surface of the substrate 310. The substrate 310 is generally a hydrophobic material except for the where the opening 320 is provided. In other words, there is either a layer of hydrophobic material (not shown) attached to an underlayment (not shown) which together forms the substrate 310, or the substrate 310 is made from a hydrophobic material. An example of the former is parchment paper, and an example of the latter is wax paper. The opening 320 can be made by performing various processing steps, e.g., shining a CO₂ laser, at the substrate 310. The substrate 310 at the opening 320 is hydrophilic. Placed in the opening 320 is an oxygen generating compound 330 that generates oxygen 160 once the oxygen-rich fluid comes in contact with it. The oxygen generating compound 330 can be a catalyst or can be part of the chemical reaction in which oxygen is released, as described further below.

Referring to FIG. 1e, a schematic for an alternative embodiment for oxygen generation platform 400 is depicted. In this embodiment, a nosepiece 420 can be worn by a subject, where the nosepiece 420 is coupled to a conduit designed to pump an oxygen-rich fluid (not shown) into the nosepiece 420 to generate a stream of oxygen vapor 430 for purposes of breathing by the subject. The oxygen vapor 430 is generated by a chemical reaction between the oxygen-rich fluid (shown) and an oxygen generating compound (not shown). The illustrated embodiment in FIG. 1e can be used as an oxygen generating and delivery platform in intensive care or emergency response situations, where the H₂O₂ is allowed to flow from a portable H₂O₂ source receptacle and into the platform 400, thereby providing the necessary O₂.

As discussed above, the substrate 310 is naturally hydrophobic; however, it can be processed to create hydrophilic spots or openings 320. The processing can be by using, for example, a CO₂ laser, or alternatively by placing a mask and acid etching the substrate 310 to expose the hydrophilic areas. This technique is applied to define an array of hydrophilic spots. An example of the oxygen generating compound 330 is MnO₂. When H₂O₂, used as oxygen-rich fluid 130, is injected through the microfluidic network 200, it reaches the spots 330, and is decomposed by the chemical catalyst (oxygen generating compound 330) for oxygen generation as shown:

2H₂O₂→2H₂O+O₂,

where H₂O is considered as a chemical byproduct which along with any unreacted/undecomposed H₂O₂ exist through the outlet 220, while the O₂ is permeated through the hydrophilic region onto the wound tissue 150.

The generated O₂ diffuses through the paper and oxygenates the wound bed below for as long as H₂O₂ flows in the microchannels 230. Biocompatible structural material allows the platform 100 to be integrated into wound healing patches which can be put in contact with wound beds to improve wound healing.

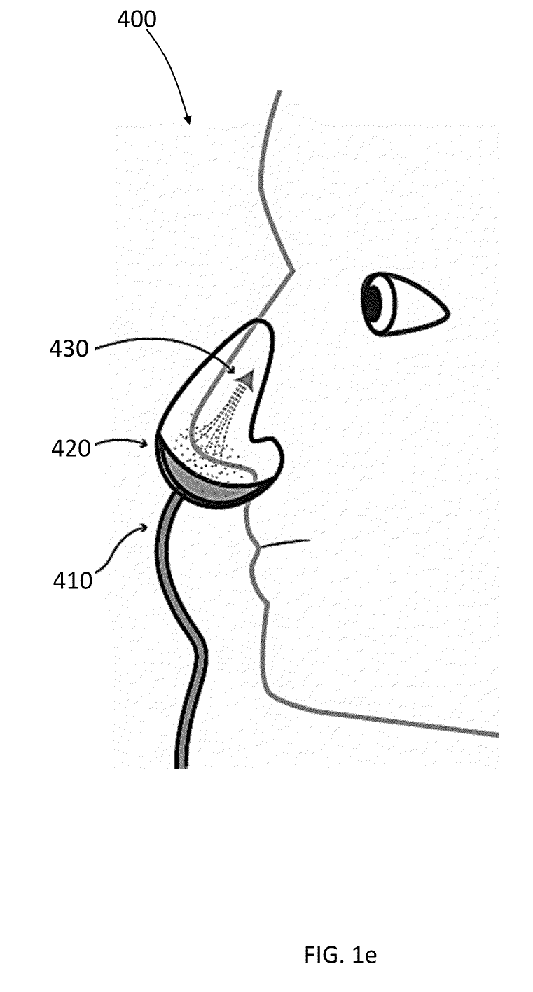

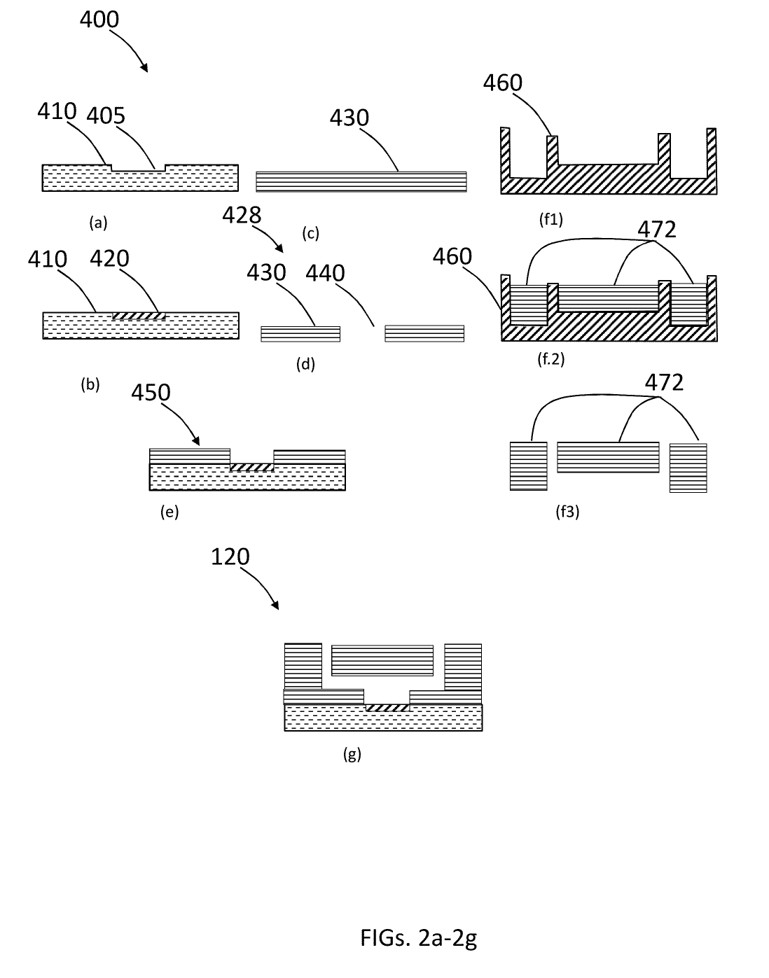

Referring to FIGS. 2a-2g, an embodiment of processing 400 of the platform 100 is depicted. The embodiment depicted in FIGS. 2a-2g, includes laser-defining patterns on a substrate 310, e.g., parchment paper, as well as creating microchannels on a polydimethyl siloxane (PDMS) layer, and then bonding all layers together. A benefit of this fabrication method is that it is relatively straightforward and requires no complex cleanroom processing. Referring to FIG. 2a, a catalyst hydrophilic pattern 405 is first laser-ablated onto a parchment paper substrate 410 that is about 30 μm thick. The paper is then dipped (for a duration of about 1 second) into a 0.1 N KMnO₄ aqueous solution followed by a dip (for a duration of about 1 second) in a 0.1 N KI aqueous solution. Referring to FIG. 2b, this dipping processes results in the deposition of KMnO₄ and KI only onto the ablated pattern. The two reactants yield MnO₂ as a catalyst 420 via the following reaction scheme:

KI(aq)+2KMnO₄(aq)+H₂O(l)→KIO₃(aq)+2KOH(aq)+2MnO₂(s)

Referring still to FIGS. 2c and 2d, a bonding step between a PDMS layer 430 that has been pattered as shown by openings 430 and the hydrophilic patterns 405 occurs through plasma-induced surface activation. Specifically, the PDMS layer 430 is spin coated on a silanized silicon wafer and cured on a hotplate (100° C., for about 20 minutes) with a final thickness of about 200 μm. The cured PDMS is transferred onto an acrylic substrate (not shown) and laser-machined to create through-hole regions 440 having the same patterns as catalyst hydrophilic pattern 405 to produce a patterned PDMS layer 428. The patterned PDMS layer is exposed to air plasma (75 W, for about 1 minute) in a plasma etcher, stamped onto uncured PDMS, and partially cured on a hotplate (65° C., for about 5 minutes). The processed PDMS layer is next bonded to the patterned parchment paper, generating a parchment paper-PDMS structure 450 by plasma-treating both the PDMS and bringing them in contact with each other, as shown in FIG. 2e.

Referring to FIGS. 2f1-2f3, 150 μm deep microchannels 472 are fabricated on a PDMS structure by casting PDMS onto a laser-machined acrylic mold 460, and the microchannels 472 are then bonded to the parchment paper-PDMS structure 450 to generate the flexible wound healing device 120, as shown in FIG. 2g.

In another embodiment, as shown in FIG. 3a, modified test platform is fabricated for characterizing the PDMS-parchment paper bond and the gas permeability of parchment paper. The test platform comprises a 14 mm×14 mm×14 mm piece of PDMS with a circular chamber of 8 mm diameter and 2 mm height. For the bond strength tests, the PDMS chamber is bonded to a piece of parchment paper either directly, with a binding layer of partially cured PDMS, or with a binding layer of uncured PDMS. In all cases, both surfaces are treated with air plasma (75 W, for about 1 minute), and then brought into contact, and finally allowed to completely cure on a hotplate at 65° C. The most favorable bonding technique (i.e., using partially cured PDMS) is then used to fabricate the test platform with the same design for characterizing the gas permeability of the parchment paper.

Referring to FIG. 3a, top view of a test PDMS-parchment paper platform 120a is depicted. The test platform 120a includes an inlet 210a, an outlet 220a, microchannels 230a, openings 320a in a substrate (not shown), and oxygen generating compound 330a sealed over the openings 320a by a layer of PDMS, fabricated in a manner similar to the processing steps shown in FIGS. 2a-2g. The platforms described herein were characterized in terms of the paper-PDMS bond strength, gas permeability, oxygen generation capability, and biocompatibility, particularly at the microchannels 230, the openings 320, and the oxygen generating compound 330. The bond strength of the PDMS-paper interface was measured using the modified test platforms. A stainless steel needle (not shown) was used for the fluid connection, and its perimeter was sealed with silicone adhesive at the inlet 210a. A syringe pump (not shown) was used to pump water into the platform at the inlet 210a at a rate of 250 μL/minute while the pressure was measured using a digital pressure gauge (DPG4000, OMEGA ENGINEERING, INC.), at various points, including the outlet 220a. The pressure just before platform failure was recorded. A similar setup was used to assess permeability of parchment paper to air. A syringe pump (not shown) was used to pump air into a test PDMS-parchment paper platform 120a bonded using partially cured PDMS and plasma. The pressure in the chamber was measured at various gas flow rates and was used to calculate the permeability of the paper.

The test PDMS-parchment paper platform 120a was also tested for oxygen generation and permeation across the parchment paper. A syringe pump (not shown) was used to drive H₂O₂ through the platform 120a to induce oxygen generation at the openings, via the oxygen generating compound 330a. A fiber-optic oxygen measurement system was used to measure the oxygen concentration on the opposite side of the parchment paper, recording the oxygen level at catalyst-free 320b and catalyst-loaded 320a openings. The oxygen level at a single spot was also monitored for 30 hours to determine the long-term generation rate. The transport kinetics of the generated oxygen was explored to determine the maximum peroxide flow rate that would permit accurate delivery of oxygen at its generation location. Oxygen generated at a spot must remain at the spot for sufficient time to allow its permeation across the parchment paper; thus, if the peroxide flow rate is too high, the generated oxygen will be transported downstream and may permeate the parchment paper at an unintended location. The effect of the liquid flow rate was determined by measuring the oxygen level (using the same fiber-optic system mentioned above) across the parchment paper at various distances from the point of generation under different flow rates.

To determine the cytotoxicity of parchment paper, 3T3 fibroblast cells with a cell density of 10×10⁴ cells/sample were seeded on the surface of the parchment paper. Because the surface of the parchment paper is hydrophobic, a short (approximately 1 minute) plasma treatment was applied before the cell seeding process. Cells with the same density were seeded onto parchment paper with catalyst, parchment paper without catalyst, and a standard well plate (which served as the control). After about six hours, alamar blue analyses were carried out to determine the cytotoxicity of the samples.

In high concentrations, H₂O₂ is known to be toxic to cells. Hence, separation of H₂O₂ flow from the cell-seeded region was verified. The test PDMS-parchment paper platform 120a was modified to include an additional 200 μm layer of PDMS bonded to the exposed parchment paper. This additional PDMS layer contained through-holes to form wells around the catalyst-loaded parchment paper regions. The wells were used both to contain and culture the cells, as well as to insure that the cells remained aligned with the oxygen-releasing spots throughout the experiment. In this experiment, the platforms were first treated with plasma. Thereafter, 3T3 fibroblast cells with a density of 5×10⁴ cells/sample were seeded on the surface of the platforms. Next, a 3% H₂O₂ solution at a flow rate 250 μl/hour was introduced through the channels for 15 hours. After the 15 hours period of culture time, alamar blue assays were performed to measure cell proliferation. As a control group, some platforms were used without any H₂O₂ flow. FIGS. 3b and 3c show photographs of the fabricated platform 120a with spots/openings 320a loaded with MnO₂.

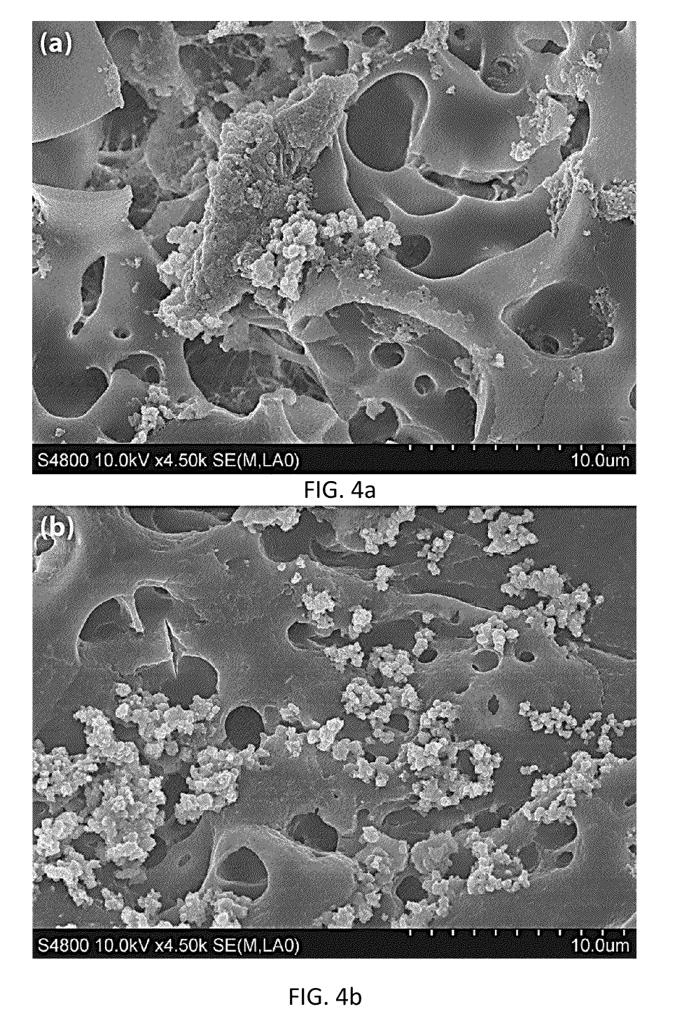

Magnified views of a catalyst spot are shown in the SEM images in FIGS. 4a and 4b. Referring to FIG. 4a, an aqueous solution of powder MnO₂ was cast on the spots whereas in FIG. 4b, the same catalyst material was deposited via a chemical reaction of two aqueous reactants (KI, KMnO₄). The images show the increased uniformity and smaller particle size achievable with the reaction-deposition approach as opposed to the powder casting method. With the reaction approach, the wicking action of the paper in the catalyst spots absorbs each of the reactants, allowing the catalyst precipitate to be generated within the paper mesh for improved particle entrapment and reduced catalyst washout rate during operation.

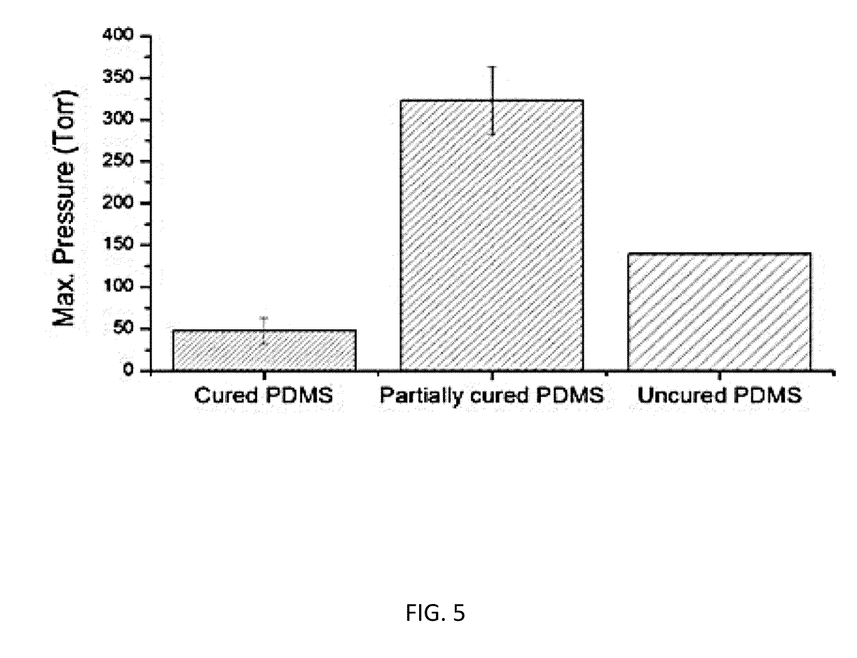

FIG. 5 depicts the characterization data for the PDMS-parchment paper bond strength. This test compared the bond strength of parchment paper bonded to PDMS according to the fabrication process described herein using fully cured PDMS, partially cured PDMS, and uncured PDMS. The results show that a maximum pressure was achieved when bonding to two materials using partially cured PDMS. This method created a bond capable of withstanding at least about 323 Torr. During these tests, the parchment paper was also observed to be impermeable to aqueous solutions for pressures below 110 Torr. Therefore, the flow of H₂O₂ in the channels was not expected to affect cell cultures on the opposite side of the parchment paper as long as the liquid pressure remained below this level.

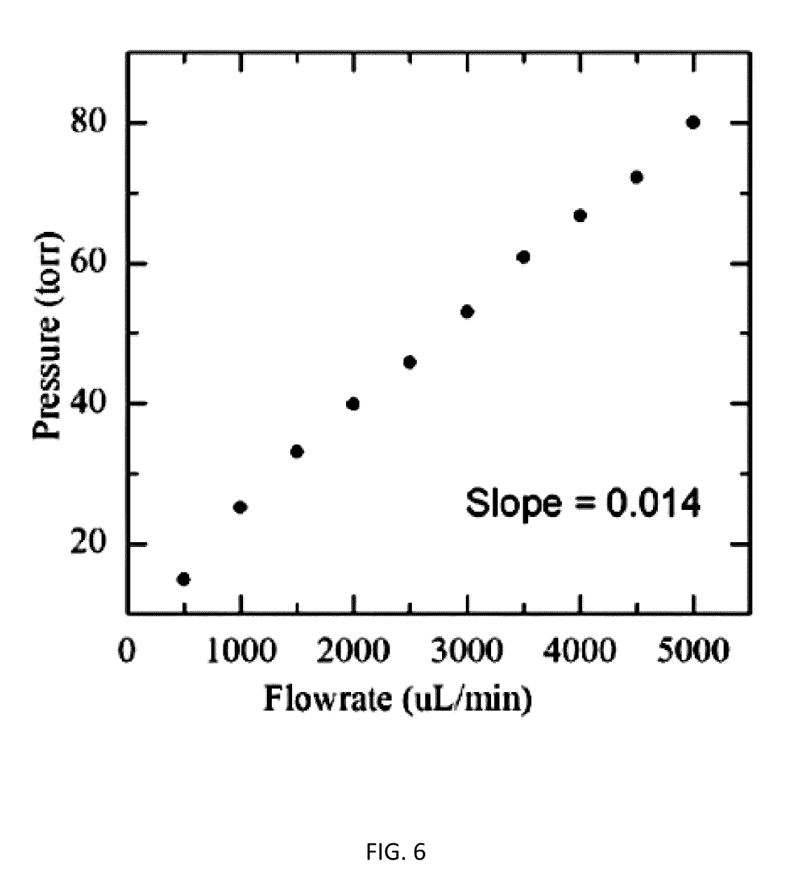

The gas permeability of parchment paper was measured using the test platform 120a, the results of which are shown in FIG. 6. The pressure (P) versus gas flow rate (Q) data reveal of slope of about 0.014 torr/μL/min. Since the test platforms had a parchment paper area (A) of 50.24 mm², the gas permeability (κ) of the paper can be computed according to Equation 1.

$$κ = \frac{Δ Q}{Δ P} \frac{1}{A} \approx 1.42 \frac{μ L}{Torr \cdot {mm}^{2} \cdot min} \qquad Equation 1$$

Assuming the maximum pressure of 110 Torr (after which H₂O₂ may permeate through the paper), the paper is suitable for oxygen generation rates of up to about 4.91 μL/min/spot. This value is sufficiently high to allow oxygen permeation at a typical wound oxygen consumption rate of about 3 mL/hour with eleven 200-μm diameter spots.

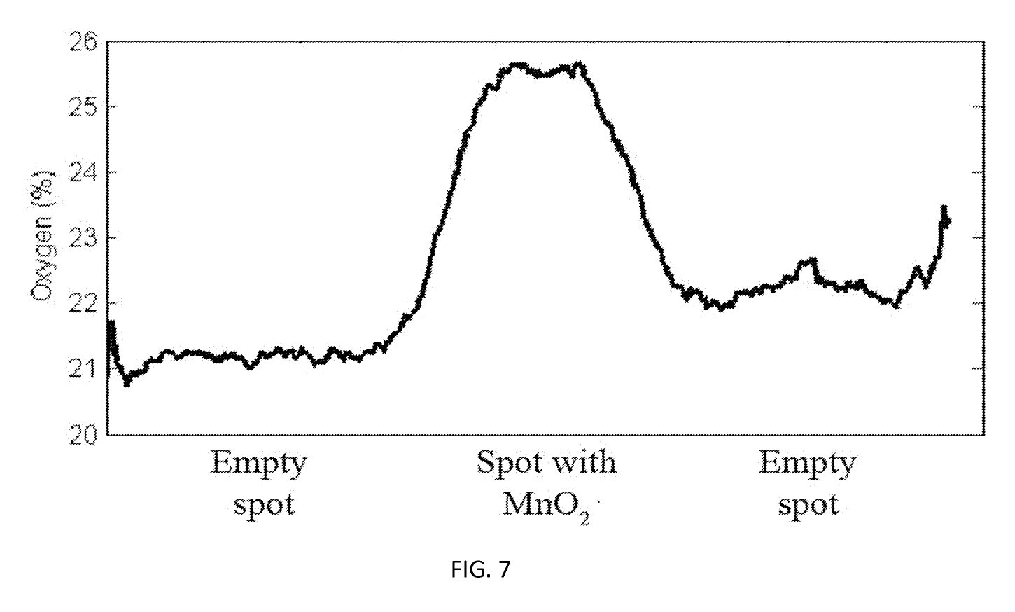

The ability to increase the oxygen level across parchment paper was confirmed with direct oxygen measurements using the optical oxygen sensor positioned 1 mm next to the paper surface. Referring to FIG. 7, a plot of oxygen % vs. various areas for an increase of oxygen concentration from about 20.9% (i.e., the relative percentage of oxygen in air) to about 25.6% (or about 4.7% higher than percentage of oxygen in air) was observed on the exposed side of the paper for regions with catalyst as compared to areas without catalyst. As seen, the areas with catalyst generate the higher level of oxygen.

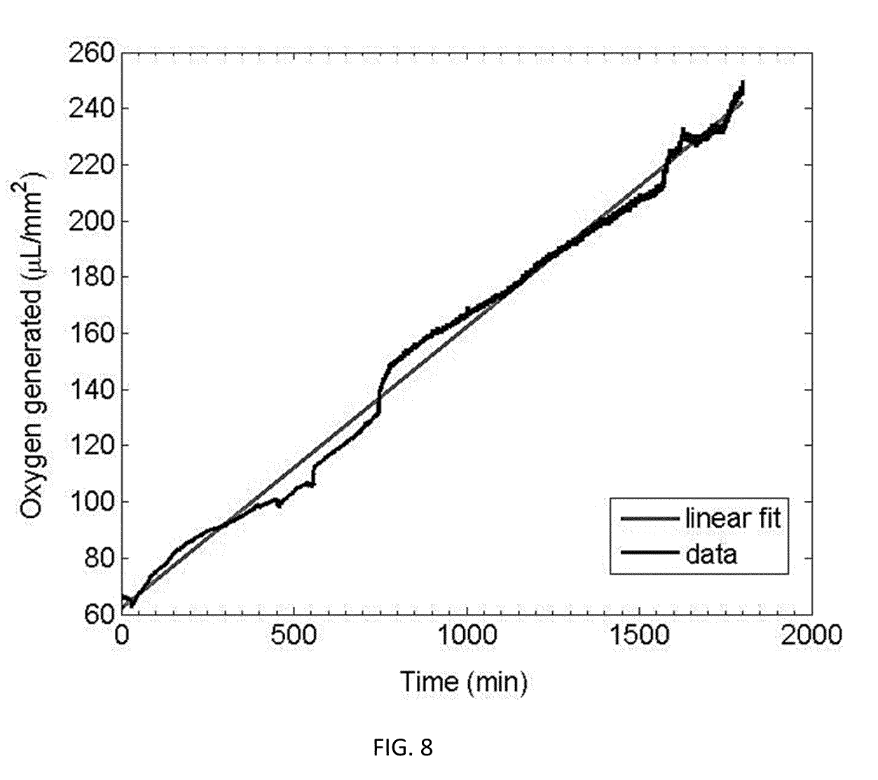

Referring to FIG. 8, a graph of Oxygen (μL/mm²) for long-term (30 h) measurements of continuous oxygen generation vs. time (min) is depicted. The graph reveals a relatively constant oxygen generation rate of about 0.1 μL O₂/min/mm² (comparison to a linear curve fit is provided). A comparable level of oxygenation (0.3 μL O₂/min/mm²) has been previously shown to effectively promote epithelial healing in a rabbit ear wound model. Thus, the platforms disclosed herein are able to generate oxygen at a sufficiently high rate to alter the oxygen level in the microenvironment of a wound and improve wound healing.

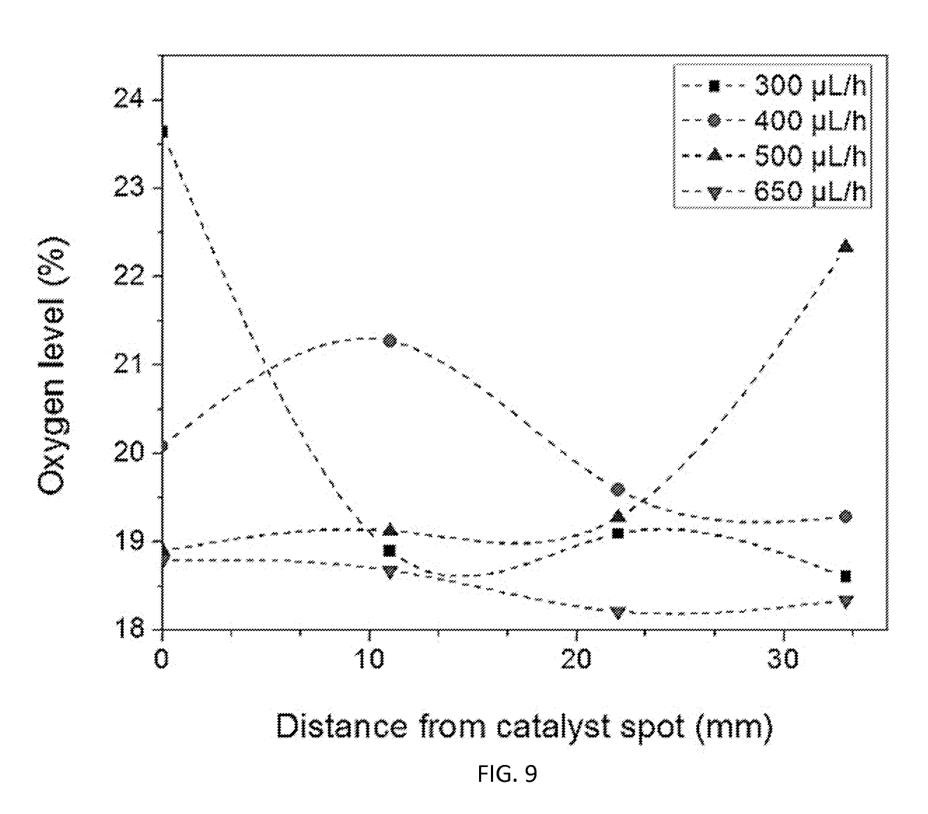

The rate of oxygenation can be further controlled by varying the amount of catalyst deposited on the spots and/or the flow rate and concentration of H₂O₂. The oxygen transport kinetics of the platforms for various flow rates are shown in FIG. 9. The plot depicts the level of oxygen as a function of the downstream distance from the point of oxygen generation for various flow rates of H₂O₂. The data show that for the channels used (rectangular cross-section of 500 μm×200 μm), a flow rate of 300 μL/h is slow enough to provide generated oxygen with sufficient time to permeate the channel and paper at the generation spot. At higher flow rates, however, cross-paper oxygen levels peak at a location downstream from the generation spot, suggesting that flow rates higher than 300 μL/h would result in excessive lateral transport of oxygen that would prevent accurate localized delivery. Therefore, the platform exhibits satisfactory performance as long as the H₂O₂ flow rate over a spot is maintained at or below 300 μL/h.

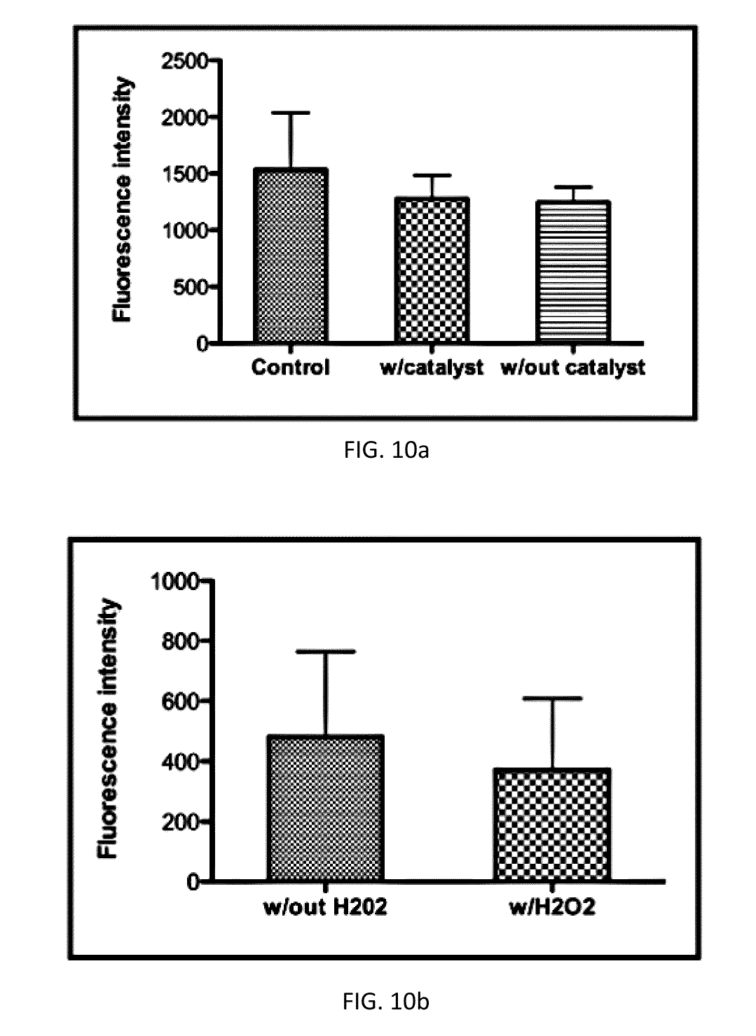

The biocompatibility results of the materials and finished platforms are shown in FIGS. 10a and 10b, where plots of fluorescence intensities are provided for areas with and without catalyst and H₂O₂. Referring to FIG. 10a, the alamar blue assay performed for 3T3 cells on parchment paper shows no significant difference between the metabolic activities of cells seeded on the culture dish as a control and that of the cells seeded on the two parchment paper samples, with and without catalyst. These results indicate the biocompatibility of both the parchment paper and the catalyst. Similarly, referring to FIG. 10b, the analyses on the assembled structures with flowing H₂O₂ showed no significant difference in the metabolic activities of the cells when compared to the control, indicating that the H₂O₂ does not come into contact with the seeded cells during platform operation and this also indicates the biocompatibility of the fabricated oxygen generators.

Other fields in medicine that will benefit from such systems are oxygen delivery to other hypoxic tissues such as cardiac, brain, tumor, etc. In addition, rapid deliveries of oxygen in emergency settings related to respiratory failure can also benefit from the current invention. Finally, many industries such as aviation require the ability to deliver oxygen in to individuals in sub-atmospheric situations, and again the herein disclosed platform can do this without the need to carry heavy and expensive oxygen supplies.

It should be noted that while parchment paper was used and described as the hydrophobic substrate, there are many other types of substrates that can be used which are biocompatible, are easily processed to generate hydrophilic regions and can be used in connection with oxygen generation scheme of the present disclosure.

While the disclosures have been illustrated and described in detail in the drawings and foregoing description, the same is to be considered as illustrative and not restrictive in character, it being understood that only certain embodiments have been shown and described and that all changes and modifications that come within the spirit of the disclosure are desired to be protected.

1. A platform for oxygen generation and delivery comprising:
a hydrophobic substrate with at least one hydrophilic region permeable to gas flow formed thereon having an oxygen generating compound embedded in the at least one hydrophilic region; and
a microfluidic network with an inlet and an outlet bonded to the hydrophobic substrate with at least one fluid exchange region fluidly coupled to the inlet and the outlet and substantially matching the least one hydrophilic region,
the microfluidic network configured to receive an oxygen rich fluid at the inlet, communicate the oxygen rich fluid to the at least one fluid exchange region to mix with the oxygen generating compound, causing a chemical reaction resulting in formation of oxygen and a chemical byproduct, and communicate the chemical byproduct to the outlet, where the oxygen is permeated out of the at least one hydrophilic region.

2. The platform of claim 1, the microfluidic network is constructed from polydimethylsiloxane (PDMS).

3. The platform of claim 1, the at least one hydrophilic region is formed using laser ablation.

4. The platform of claim 1, the embedded oxygen generating compound is a catalyst.

5. The platform of claim 4, the catalyst is MnO₂ and the oxygen rich fluid is H₂O₂.

6. The platform of claim 5, the hydrophobic substrate is parchment paper.

7. The platform of claim 6, the generated oxygen produces a concentration of at least about 4.7% higher next to the at least one hydrophilic region than the concentration of oxygen in air.

8. The platform of claim 6, oxygen is generated at a rate of about 0.1 μL O₂/min/mm².

9. The platform of claim 1, permeability for air at the at least one hydrophilic region is at least about 0.014 torr/μL/min.

10. The platform of claim 1, the bond between microfluidic network and the hydrophobic substrate can withstand at least about 323 bar fluid pressure.

11. A method of healing wounds, comprising:
placing a flexible wound healing device on a wounded tissue, the flexible wound healing device configured to generate oxygen at higher concentrations than present in air; and
injecting an oxygen-rich fluid into the flexible wound healing device,
where the flexible wound healing device includes
a hydrophobic substrate with at least one hydrophilic region formed thereon and positioned over the wounded tissue and which is permeable to gas flow and having an oxygen generating compound embedded in the at least one hydrophilic region, and
a microfluidic network with an inlet and an outlet bonded to the hydrophobic substrate with at least one fluid exchange region fluidly coupled to the inlet and the outlet substantially matching the least one hydrophilic region,
the microfluidic network configured to receive the oxygen rich fluid at the inlet, communicate the oxygen rich fluid to the at least one fluid exchange region to mix with the oxygen generating compound, causing a chemical reaction resulting in formation of oxygen and a chemical byproduct, and communicate the chemical byproduct to the outlet, where the oxygen is permeated out of the at least one hydrophilic region.

12. The method of claim 11, the embedded oxygen generating compound is a catalyst.

13. The method of claim 12, the catalyst is MnO₂ and the oxygen rich fluid is H₂O₂.

14. The method of claim 13, the hydrophobic substrate is parchment paper.

15. The method of claim 14, the generated oxygen produces a concentration of about 4.7% higher next to the at least one hydrophilic region than the concentration of oxygen in air.

16. The method of claim 14, oxygen is generated at a rate of about 0.1 μL O₂/min/mm².

17. A method of making a platform for oxygen generation and delivery, comprising:
laser-patterning a hydrophobic substrate to produce at least one hydrophilic region;
embedding an oxygen generating compound in the at least one hydrophilic region;
forming a microfluidic network having an inlet, an outlet and at least one fluid exchange region in fluid communication with the inlet and the outlet;
bonding the microfluidic network to the hydrophobic substrate such that the at least one fluid exchange region is positioned over the hydrophilic region,
where the microfluidic network is configured to receive an oxygen rich fluid at the inlet, communicate the oxygen rich fluid to the at least one fluid exchange region to mix with the oxygen generating compound, causing a chemical reaction resulting in formation of oxygen and a chemical byproduct, and communicate the chemical byproduct to the outlet, where the oxygen is permeated out of the at least one hydrophilic region.

18. The method of claim 17, the microfluidic network is constructed from polydimethylsiloxane (PDMS).

19. The method of claim 17, the hydrophobic substrate is parchment paper.

20. The method of claim 17, the step of bonding can generate a bond that can withstand at least about 323 torr of fluid pressure.
